(19) United States
(12) Patent Application Publication
(10) Pub. No.: US 2020/0211683 A1
(43) Pub. Date: Jul. 2, 2020

(54) MEDICAL INFORMATION DEVICE, MEDICAL INFORMATION SYSTEM, AND METHOD FOR MEDICAL INFORMATION PROCESSING

(71) Applicant: Konica Minolta Healthcare Americas, Inc., Wayne, NJ (US)

(72) Inventor: Hiroyuki Kubota, Wayne, NJ (US)

(73) Assignee: Konica Minolta Healthcare Americas, Inc., Wayne, NJ (US)

(21) Appl. No.: 16/236,204

(22) Filed: Dec. 28, 2018

(51) Int. Cl. G16H 15/00; G06F 17/24

(52) U.S. Cl. CPC G16H 15/00; G06F 17/248; G06F 17/243

(57) ABSTRACT

A medical information device includes a processor: causes a client device to display a first medical report template including a first input area associated with a first temporary region and a second input area associated with a second temporary region; receives a first content inputted in the first input area and a second content inputted in the second input area; stores the first content to the first temporary region, and the second content to the second temporary region; causes the client device to switch from displaying the first medical report template to displaying a second medical report template comprising a third input area associated with the first temporary region and a fourth input area associated with a third temporary region; retrieves, when the first temporary region contains the first content, the first content from the first temporary region to be displayed at the third input area.

### BACKGROUND

Electronic medical records, including medical images and other medical data play a crucial role in the diagnosis of patients. Healthcare facilities (e.g., hospitals) have realized the benefits of electronically storing medical records. The digitalization of medical images and other data not only enables users to easily access the medical images and medical data, but also enables the images and data to be easily shared between multiple healthcare facilities.

In the healthcare industry, the use of a system known as a Picture Archiving and Communications System (“PACS”) is becoming increasingly popular for convenient storage and access of medical images and reports. Generally, a PACS comprises a multitude of devices working cooperatively to digitally capture, store, manage, distribute, and display medical images generated by various imaging modalities, such as computed tomography (CT), magnetic resonance imaging (MRI), position emission tomography (PET), ultrasound, X-ray, etc. PACS allows various healthcare facilities to share all types of images and reports captured or created internally or externally.

Usually the medical reports based on those medical images are created by using a medical report template selected from a plurality of medical report templates stored in the computer. The user who creates medical reports sometimes switches from displaying the medical report template firstly selected to displaying another medical report template during drafting the medical report. This occurs in the case such that the user notices another possible cause of diseases, or the user changes his/her mind about the medical image etc. In this case of switching the medical report templates, the user needs to draft the medical report from the beginning based on the new medical report template. This process of restarting reports is a burden, and it increases the chances of user error that may cause the loss of important information in the draft report based on the previous medical report template.

### SUMMARY

One or more embodiments of this invention provide a medical information device, a medical information system, and a method for medical information processing that help the user to create a more suitable medical report.

One or more embodiments provide a medical information device including: a medical information device including: a storage that includes a first temporary region, a second temporary region, and a third temporary region; a processor that: causes a client device to display a first medical report template including a first input area associated with the first temporary region and a second input area associated with the second temporary region; receives a first content inputted in the first input area and a second content inputted in the second input area on the first medical report template being displayed; stores the first content to the first temporary region and the second content to the second temporary region; causes the client device to switch from displaying the first medical report template to displaying a second medical report template including a third input area associated with the first temporary region and a fourth input area associated with the third temporary region; confirms, when the second medical report template is displayed, whether the first temporary region associated with third input area of the second medical report template contains the first content; retrieves, when the first temporary region contains the first content, the first content from the first temporary region to be displayed at the third input area.

The processor may further: confirm, when the second medical report template is displayed, whether the second temporary region is not associated with any input areas of the second medical report template; and delete, when the second temporary region is confirmed to be not associated with any input areas of the second medical report template, the second content contained in the second temporary region.

The processor may further: confirm, when the second medical report template is displayed, whether the third temporary region associated with the fourth input area of the second medical report template contains a third content; retrieve, when the third temporary region contains the third content, the third content from the third temporary region to be displayed at the fourth input area.

The processor may further: cause the client device to switch from displaying the second medical report template to displaying a third medical report template including a fifth input area associated with the second temporary region and a sixth input area associated with the third temporary region; retrieve, when the second temporary region contains the second content, the second content from the second temporary region to be displayed at the fifth input area; and retrieve, when the third temporary region contains a third content, the third content from the third temporary region to be displayed at the sixth input area.

Each content contained in the first temporary region, the second temporary region, and the third temporary region may be deleted once a medical report is issued.

The storage may further include a first reference region, a second reference region, and a third reference region; each of the first reference region, the second reference region, and the third reference region may include: a medical-code system value; a medical-code value that is defined in a medical-code system identified by the medical-code system value; and a medical-code description that describes the medical-code value in a text format; and the first reference region, the second reference region, and the third reference region are associated with the first temporary region, the second temporary region and the third temporary region, respectively.

The processor may further create a medical report file when the processor receives an indication of completion of input, each of the first content, the second content, and a third content of a medical report include: the medical-code system value; the medical-code value; and the medical-code description.

At least one of the first content, the second content, and a third content may be a medical image stored as a link to the medical image stored in a medical image server.

One or more embodiments provide a medical information system including: a medical information device according to the above-described embodiments; and the client device that displays according to information received from the medical information device.

One or more embodiments provide a method for medical information process including: displaying a first medical report template including a first input area associated with a first temporary region of a storage and a second input area associated with a second temporary region of the storage; receiving a first content inputted in the first input area and a second content inputted in the second input area on the first medical report template displayed; storing the first content to the first temporary region, and the second content to the second temporary region; causing the client device to switch from displaying the first medical report template to displaying a second medical report template including a third input area associated with the first temporary region and a fourth input area associated with the third temporary region; confirming, when the second medical report template is displayed, whether the first temporary region associated with third input area of the second medical report template contains the first content; retrieving, when the first temporary region contains the first content, the first content from the first temporary region to be displayed at the third input area.

### BRIEF DESCRIPTION OF THE DRAWINGS

FIG. 1 shows a diagram of the medical information system according to one or more embodiments.

FIG. 2 shows a hardware diagram of the medical information device according to one or more embodiments.

FIG. 3 shows a diagram indicating storage regions of the storage of the medical information device according to one or more embodiments.

FIG. 4 shows an exemplary structure of the reference region of the storage.

FIG. 5 shows a sequence diagram of the medical information system according to one or more embodiments.

FIG. 6 shows an example of screen image shown on the display device of the client device.

FIG. 7 shows an example of screen image shown on the display device of the client device.

FIG. 8 shows an example of screen image shown on the display device of the client device.

FIG. 9 shows a flow chart for the check template process of the medical information device according to one or more embodiments.

FIG. 10 shows an example of screen image shown on the display device of the client device.

FIG. 11 shows an example of screen image shown on the display device of the client device.

FIG. 12 shows an exemplary structure of a report region.

FIG. 13 shows a flow chart for the modified check template process of the medical information device according to one or more embodiments.

FIG. 14 shows a sequence diagram of the medical information system according to one or more embodiments.

FIG. 15 shows a diagram indicating another usage of the medical information device without utilizing any networks according to one or more embodiments.

### DETAILED DESCRIPTION

Specific embodiments of the invention will now be described in detail with reference to the accompanying figures. Like elements in the various figures are denoted by like reference numerals for consistency.

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

[Medical Information System]

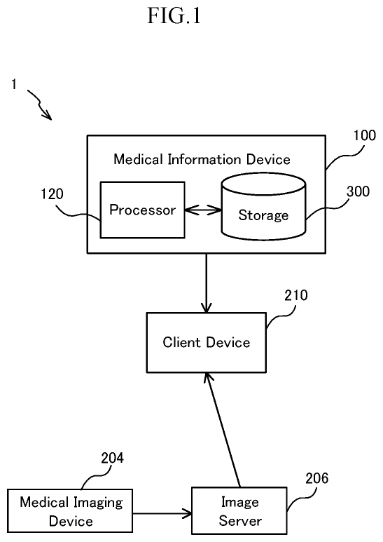

FIG. 1 shows a diagram of the medical information system 1 according to one or more embodiments. As shown in FIG. 1, the medical information system 1 includes a medical information device 100 and a client device 210 that displays screen images on the display device (not shown) according to information received from the medical information device 100. The medical information system 1 may further include a medical image server 206, and a medical imaging device 204.

The client device 210 is operated by a user to draft a medical report by accessing to the medical information device 100 through a network 170. The display device of the client device 210 can be an LCD (Liquid Crystal Display) display, an OLED (Organic Light Emitting Diode) display, a CRT (Cathode Ray Tube) display, or projection type display, etc.

The medical imaging device 204 may include devices that can capture images of computed tomography (CT), magnetic resonance imaging (MRI), position emission tomography (PET), ultrasound, X-ray, etc. The medical imaging device 204 may send their captured images to the medical image server 206 for storage. The medical images stored in the medical image server 206 may be identified by a unique identification code. The medical images stored on the medical image server 206 can be referred or retrieved as a part of the medical report on the client device 210 by using the unique identification code.

[Medical Information Device]

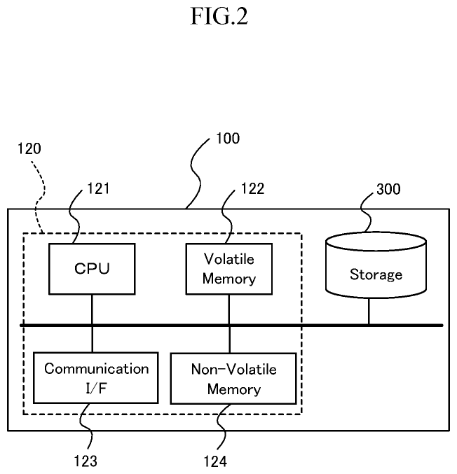

FIG. 2 shows a hardware diagram of the medical information device 100 according to one or more embodiments. As shown in FIG. 2, the medical information device 100 is an information processing device including a storage 300, a CPU (central processing unit) 121, a volatile memory 122, a non-volatile memory 124, and a communication interface 123. The storage 300 may be non-volatile memory such as hard disks, flash memories, etc. and may store medical reports. The volatile memory 122 can include RAM (random access memory), cache memory, etc. The non-volatile memory 124 can include ROM (read only memory), flash memory, hard disk, etc. The communication interface 123 connects to the network 170 to communicate with other devices, such the client device 210. In the following description, the CPU 121, the volatile memory 122, non-volatile memory, and communication interface 123 are collectively referenced as processor 120. However, it is also possible that the processor 120 includes only the CPU 121, or includes CPU 121 and any one or more of the volatile memory 122, the non-volatile memory, the communication interface 123, etc.

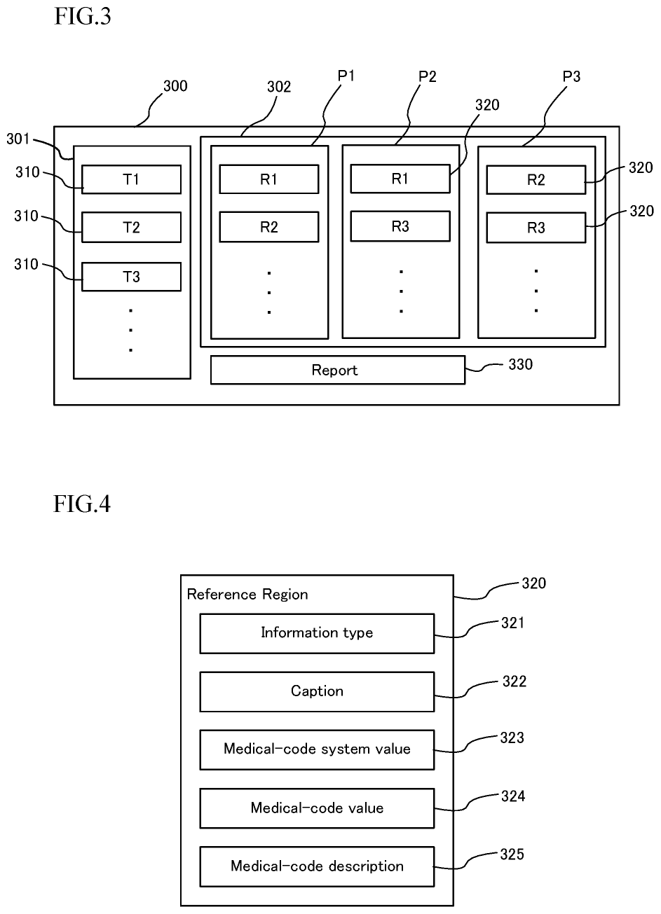

FIG. 3 shows a diagram indicating storage regions of the storage 300 of the medical information device 100 according to one or more embodiments. The storage 300 includes a contents storage 301 and a reference storage 302. The contents storage 301 includes temporary regions 310 such as a first temporary region T1, a second temporary region T2, and a third temporary region T3. The reference storage 302 may include the first medical template P1, the second medical template P2, and the third medical template P3. Any number of medical templates can be included to the reference storage 302. The user can select an appropriate medical template from the medical templates stored in the reference storage 302 to input the information based on the medical images. Each of the medical templates such as the first medical template P1, the second medical template P2, and the third medical template P3 may include reference regions 320 such as a first reference region R1, a second reference region R2, and a third reference region R3. Specifically, for example, the first medical template P1 includes the first reference region R1 and the second reference region R2. The second medical template P2 includes the first reference region R1 and the third reference region R3. The third medical template P3 includes the second reference region R2 and the third reference region R3. The first reference region R1, the second reference region R2, and the third reference region R3 are associated with the first temporary region T1, the second temporary region T2, and the third temporary region respectively. Thus, each temporary region can be identified by the information stored in the corresponding reference region. The storage 300 may further include a report region 330 that stores the completed medical reports. In FIG. 3, the contents storage 301 is located in the storage 300 as a non-volatile memory, though, the contents storage 301 can also be located in a volatile memory such as RAM.

FIG. 4 shows an exemplary structure of the reference region 320 of the storage 300. Each of the first reference region R1, the second reference region R2, and the third reference region R3 may include an information type 321, a caption 322, a medical-code system value 323, a medical-code value 324, and a medical-code description 325. The information type 321 may be related to how the content stored in the temporary region 310 is displayed after the report is completed. That is, the information type 321 may be “numeric,” “image,” “text,” “radio button,” “combo box,” etc. The caption 322 may be displayed together with the content when the medical report is displayed. The medical-code value 324 corresponds to the content stored in the temporary region 310 and is identified in the medical-code system by the medical-code system value 323. The medical-code system may be a system defined generally in public or may be a system privately defined by a user or healthcare facilities. The medical-code description 325 can be text data explaining what is indicated by the medical-code value 324 and can be displayed together with the content.

[Operation of Medical Information System]

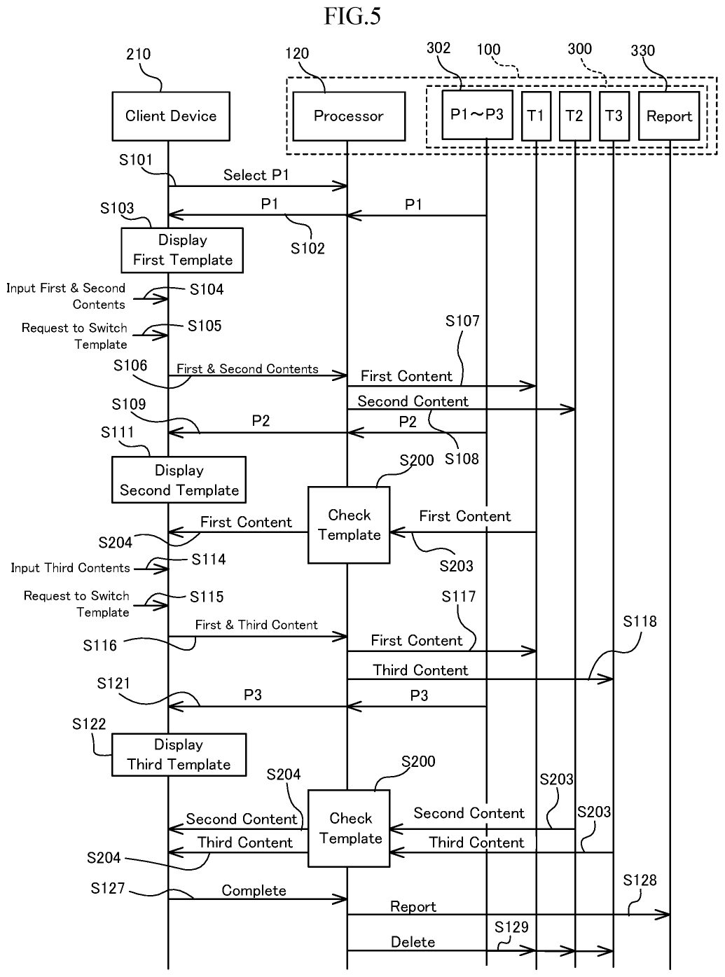

FIG. 5 shows a sequence diagram of the medical information system 1 according to one or more embodiments. As shown in this sequence diagram, according to the user's operation, firstly the client device 210 transmits the selection of the first medical report template P1 to the processor 120 of the medical information device 100 (Step S101). When the processor 120 receives the selection of the first medical report template P1, the processor 120 retrieves the first medical report template P1 from the reference storage 302 and transmits the first medical report template P1 to the client device 210 to display (Step S102). The client device 210 that receives the first medical report template P1 displays the first medical report template P1 on the display device of the client device 210. In the figures, the “medical report template” is simply written as “template” due to space limitation.

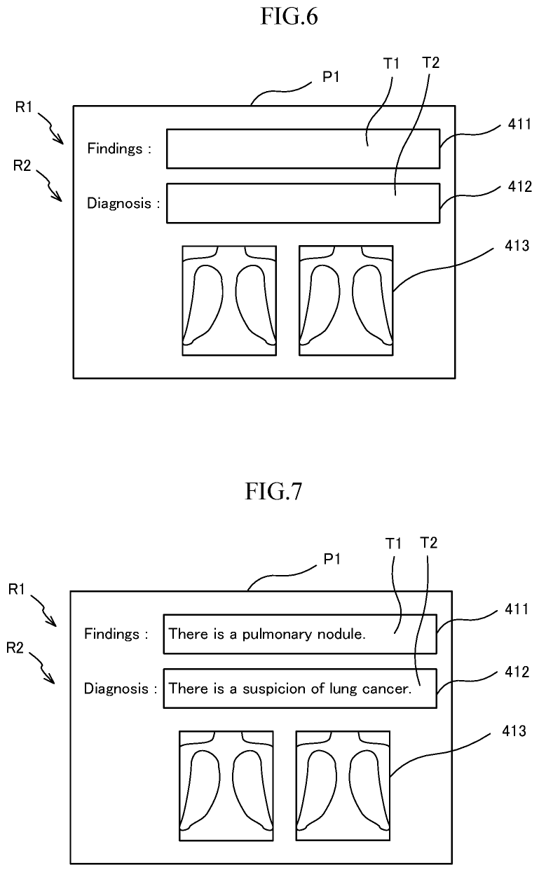

FIG. 6 shows the screen image of the first medical report template P1 displayed on the display device of the client device 210. As described above, the first medical template P1 includes the first reference region R1 and the second reference region R2. As shown in FIG. 6, the first reference region R1 of the first medical report template P1 may include the first input area 411 associated with the first temporary region T1. The second reference region R2 of the first medical report template P1 may include a second input area 412 associated with the second temporary region T2. The image input area 413 can be used for the insertion of medical images. The medical image can be stored in the storage 300 as a link to the medical image stored in a medical image server 206. Thus, the medical information device 100 can be used to create a medical report that includes medical images.

In FIG. 6, the medical images can be inserted into the image input area 413. Though, in one or more embodiments, any input areas such as the first input area 411, the second input area 412, etc. can also be used for inserting images as contents. That is, at least one of the first content and the second content can be a medical image stored as a link to the medical image stored in a medical image server 206. Also in this case, the medical information device 100 can be used for creating a medical report that includes medical images.

Referring back to FIG. 5, when the user inputs a first content into the first input area 411 and a second content into the second input area 412, the client device 210 displays the first content in the first input area 411 and the second content in the second input area 412 (Step S104). FIG. 7 shows the screen image of the first medical report template P1 when the first content and the second content are displayed in the first input area 411 and the second input area 412 respectively.

Referring back to FIG. 5, when switching from displaying the first medical report template P1 to displaying the second medical report template P2 is requested by the user of the client device 210 (Step S105), the client device 210 transmits the first content and the second content to the processor 120 of the medical information device 100 (Step S106). When the processor 120 receives a first content in the first input area 411 and a second content in the second input area 412 on the first medical report template P1, the processor 120 stores the first content to the first temporary region T1 of the storage 300 (Step S107) and the second content to the second temporary region T2 of the storage 300 (Step S108).

The processor 120 retrieves the second medical report template P2 from the reference storage 302 and transmits a second medical report template P2 to switch from displaying the first medical report template P1 to displaying a second medical report template P2 on the display device of the client device 210 (Step S109). When the client device 210 receives the second medical report template P2, the client device 210 displays the second medical report template P2 on the display device (Step S111).

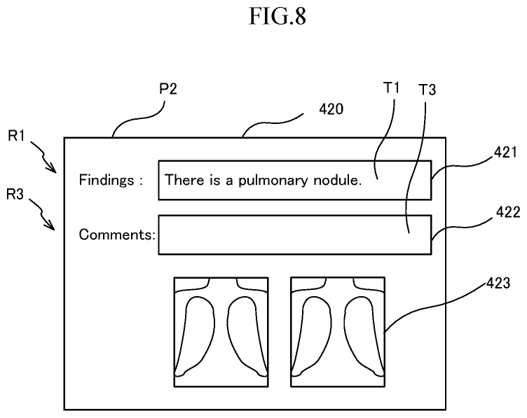

FIG. 8 shows the screen image of the second medical report template P2. As described above, the second medical template P2 includes the first reference region R1 and the third reference region R3. The first reference region R1 of the second medical report template P2 includes a third input area 421 associated with the first temporary region T1. The third reference region R3 of the second medical report template P2 may include a fourth input area 422 associated with the third temporary region T3. After transmitting the second medical report template P2, the processor 120 of the medical information device 100 executes the check template process S200.

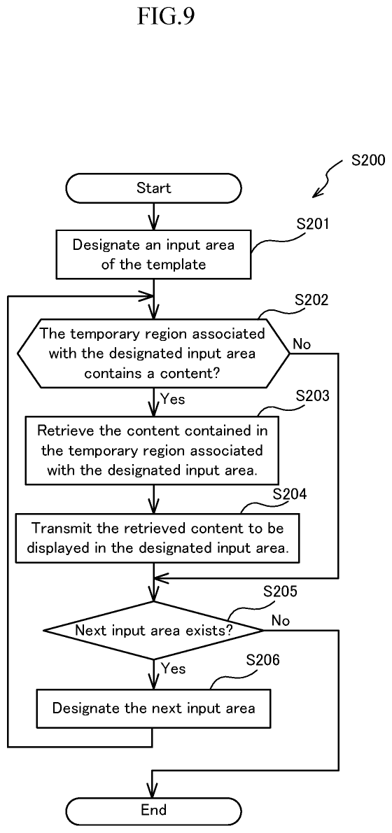

FIG. 9 shows a flow chart for the check template process S200 of the medical information device 100 according to one or more embodiments. As shown in FIG. 9, the processor 120 firstly designates an input area of the new medical report template, e.g. the third input area 421 of the second medical report template P2 (Step S201). Next, the processor 120 confirms whether the temporary region associated with the designated input area contains a content. That is, the processor 120 confirms whether the first temporary region T1 associated with third input area 421 of the second medical report template P2 contains the first content (Step S202). When the first temporary region T1 does not contain any content (Step S202: No), the process proceeds to Step S205.

When the first temporary region T1 contains the first content (Step S202: Yes), the processor 120 retrieves the content contained in the temporary region associated with the designated input area. That is, the processor 120 retrieves the first content from the first temporary region T1 (Step S203), and transmits the retrieved first content to be displayed at the third input area 421 (Step S204). At the next Step S205, the processor confirms whether an input area that has not been designated yet exists. When an input area that has not been designated yet exists (Step S205: Yes), the processor designates the next undesignated input area (Step S206) and returns the process to Step S202. When all the input areas have been designated (Step S205: No), the check template process S200 is ended. The first content inserted in the third input area 421 is shown in the screen image of FIG. 8.

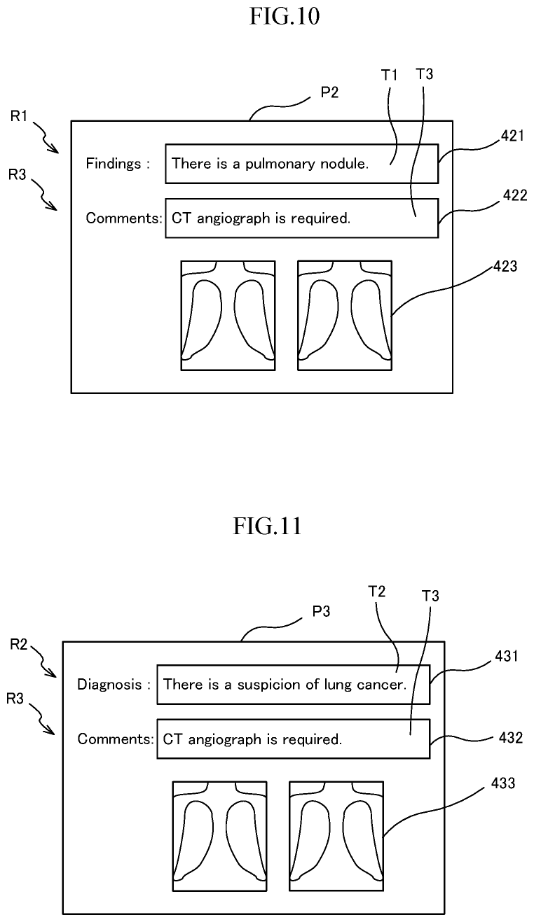

Referring back to FIG. 5, when a third content is inputted to the fourth input area 422 of the second medical report template P2 by the user, the client device 210 displays the third content in the fourth input area 422 (Step S114). FIG. 10 shows the screen image of the second medical report template P2 when the third content in the fourth input area 422 is displayed.

Referring back to FIG. 5, when the switch request from displaying the second medical report template P2 to displaying the third medical report template P3 is instructed by the user of the client device 210 (Step S115), the client device 210 transmits the first content and the third content to the processor 120 of the medical information device 100 (Step S116). When the processor 120 receives a first content in the third input area 421 and a third content in the fourth input area 422 on the second medical report template P2, the processor 120 stores the first content to the first temporary region T1 of the storage 300 (Step S117) and the third content to the third temporary region T3 of the storage 300 (Step S118).

The processor 120 retrieves the third medical report template P3 from the reference storage 302 and transmits a third medical report template P3 to switch from displaying the second medical report template P2 to displaying a third medical report template P3 on the display device of the client device 210 (Step S121). When the client device 210 receives the third medical report template P3, the client device 210 displays the third medical report template P3 on the display device (Step S122).

FIG. 11 shows the screen image of the third medical report template P3. As described above, the third medical template P3 includes the second reference region R2 and the third reference region R3. The second reference region R2 of the third medical report template P2 may include a fifth input area 431 associated with the second temporary region T2. The third reference region R3 of the third medical template P3 may include a sixth input area 432 associated with the third temporary region T3. After transmitting the third medical report template P3, the processor 120 of the medical information device 100 executes the check template process S200.

Since the check template process S200 shown in FIG. 9 is the same as previously explained, the duplicate description is omitted. As a result of the execution of the check template process S200, the second content is inserted in the fifth input area 431 and the third content is inserted in the sixth input area 432 as shown in the screen image of FIG. 11. Thus, the user can create the medical report more effectively since the user can recycle the contents contained in not only the previous template also the templates before the previous template.

Referring back to FIG. 5, when the completion of drafting the medical report is instructed by the user of the client device 210, the second content and the third content are transmitted to the processor 120 (Step S127). The processor 120 creates the medical report as an electronic file by combining the contents such as the second content, the third content, and information stored in the reference region 320. The processor 120 stores the medical report to the report region 330 (Step S128). At the end, the processor 120 deletes the remaining contents stored in the first temporary region T1, the second temporary region T2, and the third temporary region T3 once the medical report is issued (Step S129). Thus, the medical information device 100 can prevent the user from using old contents in a new and possibly unrelated report and preserve the security of private information.

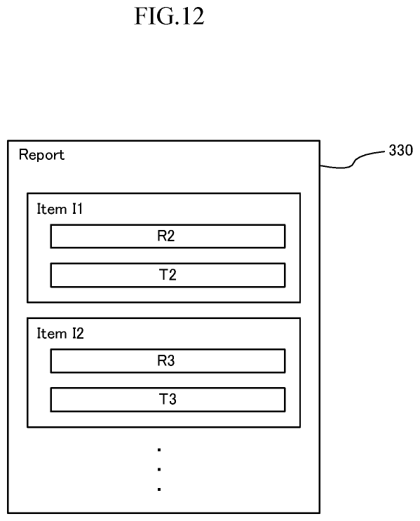

FIG. 12 shows an exemplary structure of a report region 330. The report region includes the plurality of items such as an item I1, and an item 12. The plurality of items may be arranged in an order to be displayed. The item I1 contains a region for storing the second content and the second reference region R2. The item 12 contains a region for storing the third content and the third reference region R3. Since the report region 330 includes the reference region for each content as a group belonging to an item, the reference region allows the processor 120 to determine whether the content should be displayed, with what style the item should be displayed, etc. Also since the reference regions are disposed adjacent to the corresponding contents, the processor 120 can recognize the reference region by reading one way from the top or the bottom of the report region 330.

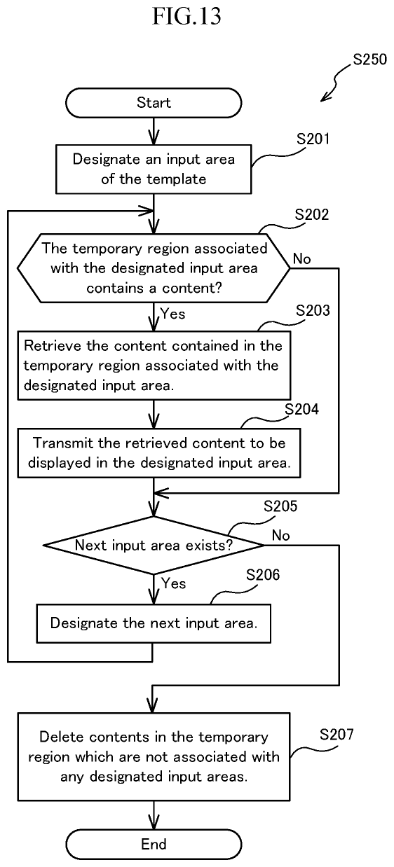

FIG. 13 shows a flow chart for the modified check template process S250 of the medical information device 100. Compared with the check template process S200 shown in FIG. 9, only Step S207 is added to the check template process S200 in FIG. 9 and the other steps are the same as the check template process S200. The duplicated explanations for the other steps are omitted. In the modified check template process S250, when all the input areas have been designated (Step S205: No), the processor 120 deletes the contents in the temporary regions which are not associated with any designated input areas.

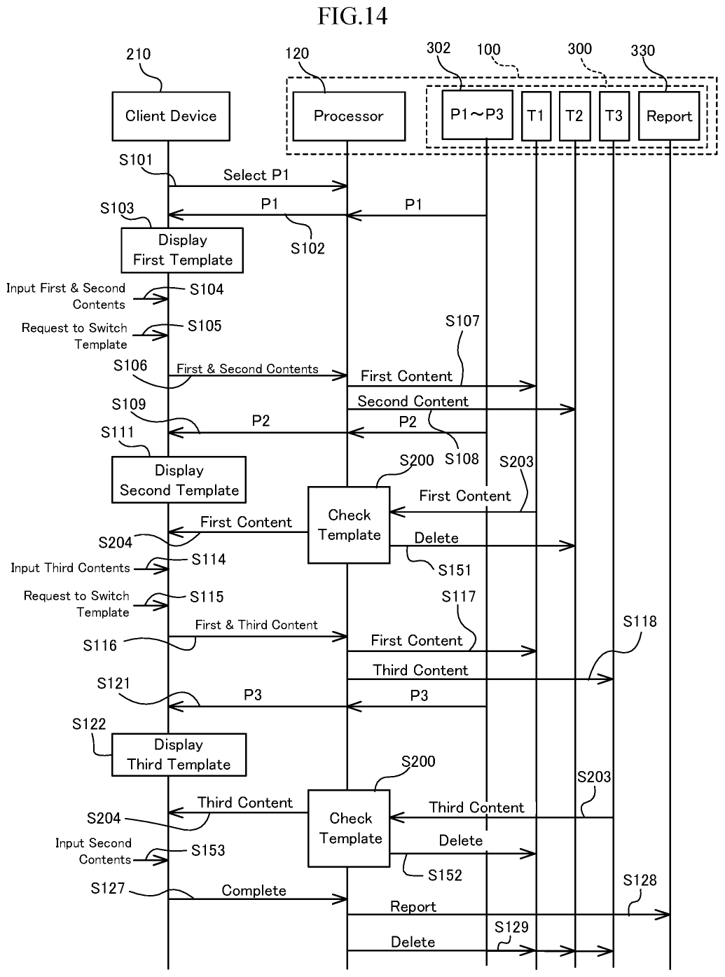

FIG. 14 shows a sequence diagram of the medical information system 1 by using the modified check template process S250 shown in FIG. 13 according to one or more embodiments. Since most of the processes are the same processes as shown in the sequence diagram in FIG. 5, the duplicated explanations for the same processes are omitted. As shown in FIG. 14, in the modified check template process S250 relating to the second medical report template P2, when the second temporary region T2 is confirmed to be not associated with any input areas of the second medical report template P2, the processor 120 deletes the second content contained in the second temporary region T2 (Step S151).

Further, when the third medical report template P3 is displayed, only the third content is inserted in the sixth input area 432 as a result of the execution of the check template process S250 (S204). The first temporary region T1 is confirmed to be not associated with any input areas of the third medical report template P3, the processor 120 deletes the first content contained in the first temporary region T1 (Step S152). Thus, since the new template does not reflect the old information stored in the template before the previous template, the medical information device 100 can prevent the user from using old or incorrect contents. The second content is entered into the fifth input area 431 of the third medical report template P3 by the user (Step S153).

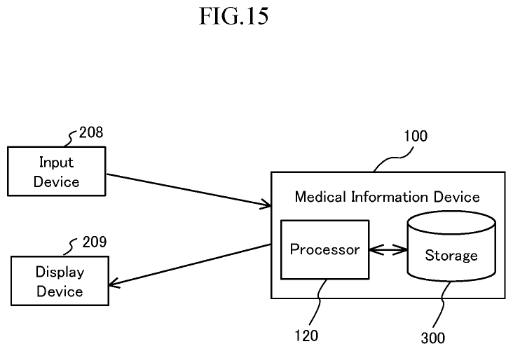

FIG. 15 shows a diagram indicating another usage of the medical information device 100. In the medical information system 1 shown in FIG. 1, the processor 120 of the medical information device 100 transmits the medical report templates and the contents to the client device 210 through the network 170. However, the processor 120 of the medical information device 100 may transmit the medical report templates to the display device 209 directly and receive contents from the internal memory of the medical information device 100 without utilizing any communication networks, that is stand-alone use. The processor 120 of the medical information device 100 may also receive the contents from the input device 208—such as a keyboard, a mouse, or a touch panel. The same advantageous effects could be obtained even in the configuration shown in FIG. 15. The contents storage 301 can be located in a non-volatile memory such as a flash memory or hard disks in the medical information device 100. The contents storage 301 can also be located in a volatile memory such as RAM.

One or more embodiments of the present invention provide the following technological improvements in computer functionality. One or more embodiments of the present invention are able to reserve the inputted contents even if the medical report templates have been switched many times. Thus, since the content that has already inputted by the user cannot be lost, the user can easily switch the medical report templates to more suitable one. Therefore, the medical information device 100 can help the user to create a more suitable medical report. Further, the storage of such data as medical-code, description, and others within the reference region 320 of one or more embodiments facilitates the introduction of new medical report templates within the medical information device 100. When a new medical report template is introduced, the medical information device 100 can easily associate the input areas of the new medical report templates with the existing temporary region, using the information stored in the reference region 320. Thus, since one or more embodiments of the present invention can easily accept new medical report templates, one or more embodiments of the present invention can help the user to create a more suitable medical report. Additionally, since one or more embodiments of the present invention can delete all the contents in the temporary regions once the medical report is issued, the contents can be deleted more securely. Thus, private information is securely protected.

Although the disclosure has been described with respect to only a limited number of embodiments, those skilled in the art, having benefit of this disclosure, will appreciate that various other embodiments may be devised without departing from the scope. Accordingly, the scope of the invention should be limited only by the attached claims.

1. A medical information device comprising:
a storage that comprises a first temporary region, a second temporary region, and a third temporary region;
a processor that:
causes a client device to display a first medical report template comprising a first input area associated with the first temporary region and a second input area associated with the second temporary region;
receives a first content inputted in the first input area and a second content inputted in the second input area on the first medical report template being displayed;
stores the first content to the first temporary region and the second content to the second temporary region;
causes the client device to switch from displaying the first medical report template to displaying a second medical report template comprising a third input area associated with the first temporary region and a fourth input area associated with the third temporary region;
confirms, when the second medical report template is displayed, whether the first temporary region associated with third input area of the second medical report template contains the first content;
retrieves, when the first temporary region contains the first content, the first content from the first temporary region to be displayed at the third input area.

2. The medical information device according to claim 1, wherein the processor further:
confirms, when the second medical report template is displayed, whether the second temporary region is not associated with any input areas of the second medical report template; and
deletes, when the second temporary region is confirmed to be not associated with any input areas of the second medical report template, the second content contained in the second temporary region.

3. The medical information device according to claim 1, wherein the processor further:
confirms, when the second medical report template is displayed, whether the third temporary region associated with the fourth input area of the second medical report template contains a third content;
retrieves, when the third temporary region contains the third content, the third content from the third temporary region to be displayed at the fourth input area.

4. The medical information device according to claim 1, wherein the processor further:
causes the client device to switch from displaying the second medical report template to displaying a third medical report template comprising a fifth input area associated with the second temporary region and a sixth input area associated with the third temporary region;
retrieves, when the second temporary region contains the second content, the second content from the second temporary region to be displayed at the fifth input area; and
retrieves, when the third temporary region contains a third content, the third content from the third temporary region to be displayed at the sixth input area.

5. The medical information device according to claim 1, wherein each content contained in the first temporary region, the second temporary region, and the third temporary region is deleted once a medical report is issued.

6. The medical information device according to claim 1, wherein
the storage further comprises a first reference region, a second reference region, and a third reference region;
each of the first reference region, the second reference region, and the third reference region comprises:
a medical-code system value;
a medical-code value that is defined in a medical-code system identified by the medical-code system value; and
a medical-code description that describes the medical-code value in a text format; and
the first reference region, the second reference region, and the third reference region are associated with the first temporary region, the second temporary region and the third temporary region, respectively.

7. The medical information device according to claim 6, wherein:
the processor further creates a medical report file when the processor receives an indication of completion of input,
each of the first content, the second content, and a third content of a medical report comprises:
the medical-code system value;
the medical-code value; and
the medical-code description.

8. The medical information device according to claim 1, wherein at least one of the first content, the second content, and a third content is a medical image stored as a link to the medical image stored in a medical image server.

9. A medical information system comprising:
a medical information device according to claim 1; and
the client device that displays according to information received from the medical information device.

10. A method for medical information process comprising:
displaying a first medical report template comprising a first input area associated with a first temporary region of a storage and a second input area associated with a second temporary region of the storage;
receiving a first content inputted in the first input area and a second content inputted in the second input area on the first medical report template displayed;
storing the first content to the first temporary region, and the second content to the second temporary region;
causing the client device to switch from displaying the first medical report template to displaying a second medical report template comprising a third input area associated with the first temporary region and a fourth input area associated with the third temporary region;
confirming, when the second medical report template is displayed, whether the first temporary region associated with third input area of the second medical report template contains the first content;
retrieving, when the first temporary region contains the first content, the first content from the first temporary region to be displayed at the third input area.
